(19) United States
(12) Patent Application Publication
(10) Pub. No.: US 2009/0110783 A1
(43) Pub. Date: Apr. 30, 2009

(54) MICROWAVEABLE EDAMAME BOWL

(76) Inventor: Jing-Yau Chung, Houston, TX (US)

Correspondence Address: MARK A OATHOUT, 3701 KIRBY DRIVE, SUITE 960, HOUSTON, TX, 77098, US

(21) Appl. No.: 12/258,143

(22) Filed: Oct. 24, 2008

Related U.S. Application Data
(60) Provisional application No. 60/982,226, filed on Oct. 24, 2007.

(51) Int. Cl. B65D 81/34; G01N 33/02

(52) U.S. Cl. 426/107; 426/232

(57) ABSTRACT

Edamame within a microwave bowl is introduced for preparation of an edamame food product. The pod count of the edamame product is within a range of about 145 to 170 pods per 500 grams of the edamame product. A volume of moisture is added into the microwaveable bowl with the edamame product. A top film is sealed across a rim of the microwaveable bowl and then the edamame product is frozen. Subsequently several sealed microwaveable bowls are packaged together in a box.

### CROSS-REFERENCE TO RELATED APPLICATIONS

This application claims the benefit of U.S. provisional application No. 60/982,226 filed Oct. 24, 2007.

### STATEMENTS REGARDING FEDERALLY SPONSORED RESEARCH OR DEVELOPMENT

Not Applicable.

### BACKGROUND

In the past, pre-cooked edamame was placed in a bag and frozen. When cooked on a normal plate it tends to lose its moisture and become dry.

### SUMMARY

Edamame within a microwave bowl is introduced for preparation of an edamame food product. The pod count of the edamame product is within a range of about 145 to 170 pods per 500 grams of the edamame product. A volume of moisture is added into the microwaveable bowl with the edamame product. A top film is sealed across a rim of the microwaveable bowl and then the edamame product is frozen.

For purpose of this application a “microwaveable bowl” shall mean any semi-rigid or rigid bowl or tray approved for preparation of a food product via electromagnetic radiation.

### BRIEF DESCRIPTION OF THE DRAWINGS

FIG. 1 is a perspective view of one embodiment of an edamame bowl.

FIG. 2 is a perspective view of several edamame bowl embodiments in packaged configuration.

FIG. 3 is a perspective view of several edamame bowl embodiments in the packaged configuration and partially housed in a box.

FIG. 4 is a perspective view of an embodiment of an edamame bowl.

FIG. 5 is a perspective view of several edamame bowl embodiments in packaged configuration.

FIG. 6 is a perspective view of several edamame bowl embodiments in the packaged configuration and partially housed in a box.

FIG. 7 is a perspective view of an embodiment of an edamame bowl.

FIG. 8 is a perspective view of several edamame bowl embodiments in packaged configuration.

FIG. 9 is a perspective view of several edamame bowl embodiments in the packaged configuration and partially housed in a box.

FIG. 10 is a perspective view of edamame beans and pods.

### DESCRIPTION OF THE PREFERRED EMBODIMENT(S)

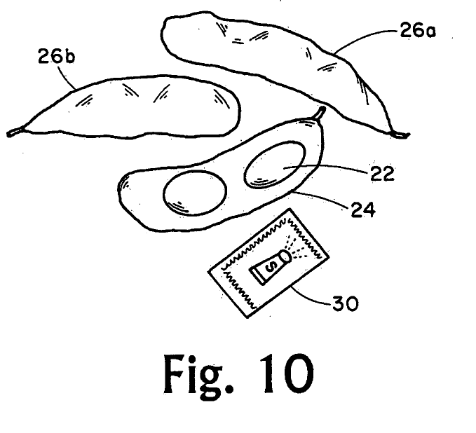

Referring to the several figures of the drawings, edamame within a microwave bowl 10 is introduced in this disclosure including methods pertaining thereto. The edamame product 20 (FIG. 10) is placed in a microwaveable bowl 10, next sealed with a top film (e.g. plastic) 12 and then frozen. This combination locks moisture within the bowl 10, enables steam cooking of the edamame, and maintains the fresh appearance of the edamame. Moreover, the edamame within a microwave bowl 10 is easy for consumers to tote (for a lunch, snack, etc.).

The microwaveable bowl 10 is preferably rectangular (as shown). Other shapes may be used. The microwaveable bowl 10 has a rim 11. However the rectangular microwaveable bowl 10 affords certain advantages in the packaging process.

A “good” quality seal is important. In order to achieve a proper seal across the rim 11 of the microwaveable bowl 10 it is critical to select good quality edamame. For purposes of this application, “good quality” edamame is that which normally would fall within a pod count range of 160 to 170 pods for 500 grams of edamame. Good quality edamame may also include a pod count range of 145 to 160 pods for 500 grams of edamame whenever such a grade of edamame is available. However, for example, a pod count range of 180 pods for 500 grams of edamame can result in a “bad” seal (e.g. the increased edamame shell volume may interfere with a proper seal).

One of ordinary skill in the art understands how to accomplish the seal of the top film 12 across the microwaveable bowl 10.

Water, moisture, or any other suitable fluid may be added to the microwaveable bowl 10 or the edamame within the microwaveable bowl 10 prior to freezing. It has, in fact, been discovered to be advantageous to add water to the natural amount of moisture carried by the edamame pod in order to enhance the final cooked edamame product. By way of example only, water may be sprayed onto the edamame pods within the microwaveable bowl 10 prior to freezing or water may be sprayed onto the edamame pods prior to the first stage blast freezing the edamame prior to the final packaging into the microwavable bowl 10.

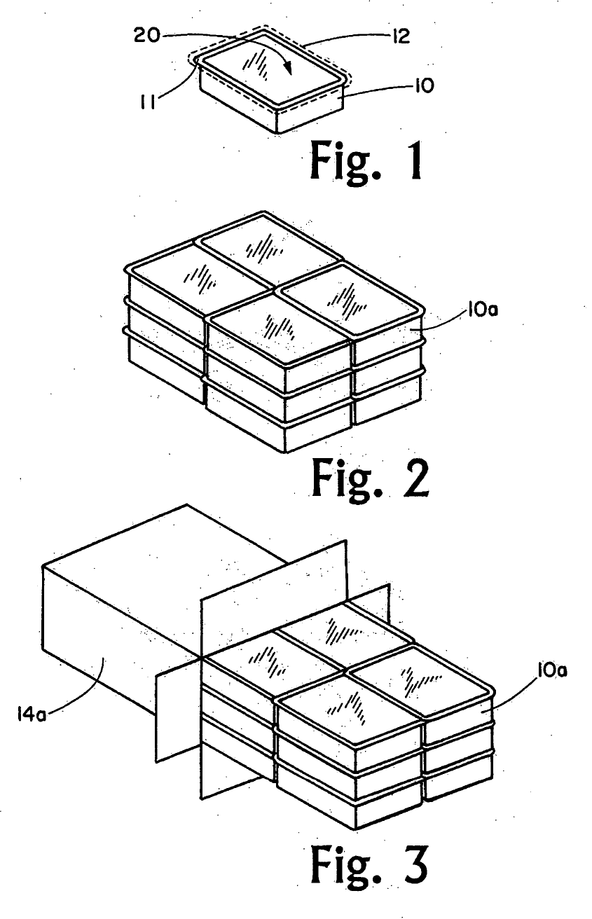

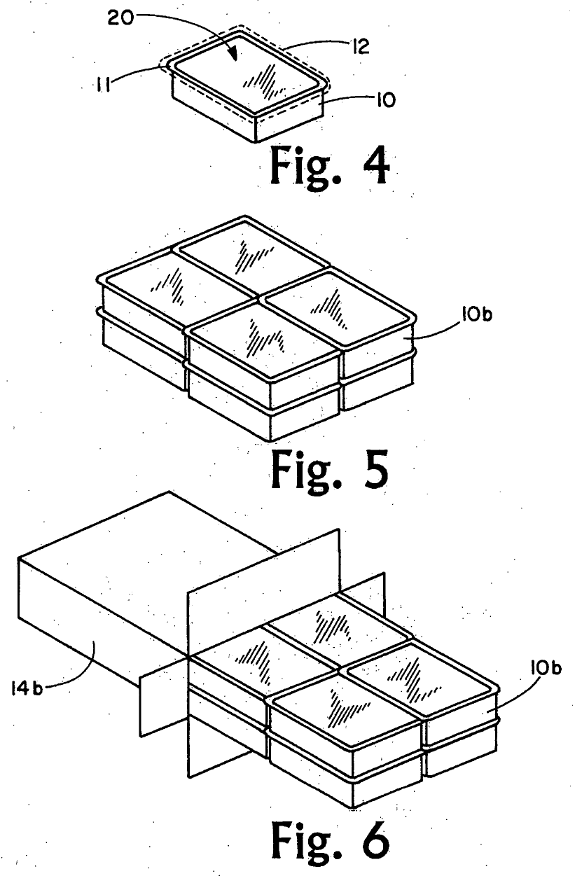

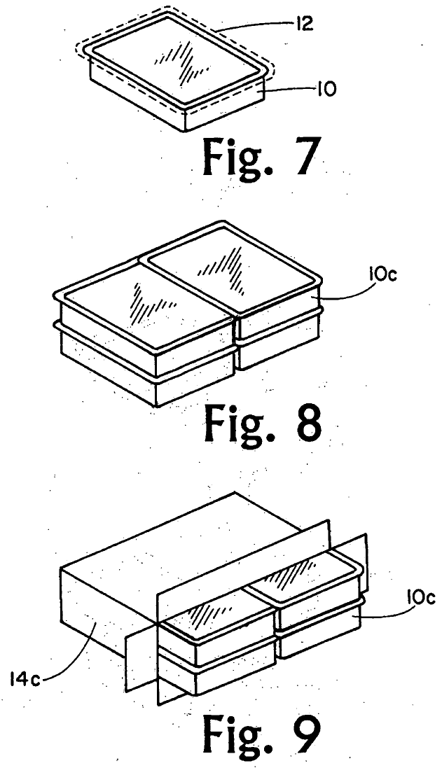

Several examples of packaging include, first, twelve microwaveable bowls 10a placed in a box 14a (see FIGS. 1-3). In this case the size of each microwaveable bowl 10a may be 5.5 inches by 4.7 inches by 1.75 inches for containing eight ounces of edamame product. Next, eight microwaveable bowls 10b may be placed in a box 14b (see FIGS. 4-6). In this case the size of each microwaveable bowl 10b may be 5.5 inches by 4.7 inches by 1.75 inches for containing eight ounces of edamame product. In another example, four microwaveable bowls 10c may be placed in a box 14c (see FIGS. 7-9). In this case the size of each microwaveable bowl 10c may be 5.5 inches by 4.7 inches by 1.45 inches for containing six ounces of edamame product, or 5.5 inches by 4.7 inches by 1.2 inches for containing five ounces of edamame product.

Currently, it is preferred to leave the edamame beans 22 within the shell 24 in the embodiment(s) described (i.e. sold to consumers as a pod 26) as the edamame product 20. However, the shell 24 could be removed prior to placing the beans 22 within the microwaveable bowl 10. Other vegetables, for example corn or peas, could be mixed with or replace the edamame within the microwaveable bowl 10.

The edamame product 20 may be blanched for a short time in the manufacturing process by any known blanching process. This may make the edamame product greener and moister. By way of example, the edamame product 20 may be blanched for less than one hundred-and-twenty seconds (ninety seconds being a representative preferred time for duration of blanching). If the duration of blanching is too long, e.g. one hundred-and-twenty to one hundred-and-fifty seconds, then the microwave radiation can make the food product overly dry.

A separate salt packet 30 (FIG. 10) such as that containing sea salt may be placed within the microwaveable bowl 10 or other packaging.

What is claimed is:

1. A microwaveable apparatus for preparation of an edamame food product, comprising:
a microwaveable bowl including a rim;
an edamame product located in the microwaveable bowl, wherein said edamame product has a pod count range of about 145 to 170 pods per 500 grams of said edamame product;
a volume of fluid added in the microwaveable bowl with said edamame product; and
a top film sealed across the rim of the microwaveable bowl.

2. The apparatus according to claim 1, wherein said edamame product has a pod count range of about 160 to 170 pods per 500 grams of said edamame product.

3. The apparatus according to claim 1, wherein said edamame product is a blanched edamame product.

4. The apparatus according to claim 1, wherein the volume of fluid is a volume of water.

5. The apparatus according to claim 4 wherein the volume of water is a spray of water applied to said edamame product.

6. The apparatus according to claim 4 wherein the volume of water is a spray of water applied in the microwaveable bowl.

7. The apparatus according to claim 1 wherein the microwaveable bowl comprises a total of twelve microwaveable bowls for placement in a box; wherein each of the twelve microwaveable bowls has dimensions of 5.5 inches by 4.7 inches by 1.75 inches; and wherein each of the twelve microwaveable bowls includes eight ounces of edamame product.

8. The apparatus according to claim 1 wherein the microwaveable bowl comprises a total of eight microwaveable bowls for placement in a box; wherein each of the eight microwaveable bowls has dimensions of 5.5 inches by 4.7 inches by 1.75 inches; and wherein each of the eight microwaveable bowls includes eight ounces of edamame product.

9. The apparatus according to claim 1 wherein the microwaveable bowl comprises a total of four microwaveable bowls for placement in a box; wherein each of the four microwaveable bowls has dimensions of 5.5 inches by 4.7 inches by 1.45 inches; and wherein each of the four microwaveable bowls includes six ounces of edamame product.

10. The apparatus according to claim 1 wherein the microwaveable bowl comprises a total of four microwaveable bowls for placement in a box; wherein each of the four microwaveable bowls has dimensions of 5.5 inches by 4.7 inches by 1.2 inches; and wherein each of the four microwaveable bowls includes five ounces of edamame product.

11. The apparatus according to claim 1 further comprising a salt packet inserted into the microwaveable bowl.

12. The apparatus according to claim 1 wherein said edamame product comprises a plurality of edamame beans having no shell.

13. A microwaveable apparatus for preparation of an edamame food product, comprising:
a microwaveable bowl including a rim;
an edamame product located in the microwaveable bowl, wherein said edamame product is a blanched edamame product, and wherein said edamame product has a pod count range of about 145 to 170 pods per 500 grams of said edamame product;
a volume of water added in the microwaveable bowl with said edamame product; and
a top film sealed across the rim of the microwaveable bowl.

14. A method for preparing a microwaveable edamame food product, comprising the steps of:
placing the edamame product in a microwaveable bowl including determining a pod count ranging from about 145 to 170 pods per 500 grams of the edamame product;
adding moisture to the edamame product;
sealing the edamame product in the microwaveable bowl; and
freezing the edamame product.

15. The method according to claim 14 wherein said step of placing the edamame product in the microwaveable bowl comprises placing the edamame product in the microwaveable bowl including determining the pod count ranging from about 160 to 170 pods per 500 grams of the edamame product.

16. The method according to claim 14 wherein said step of adding moisture to the edamame product comprises spraying water onto the edamame product.

17. The method according to claim 14 further comprising the step of blanching the edamame product.

18. The method according to claim 17 wherein said step of blanching the edamame product comprises blanching for less than one hundred-and-twenty seconds.

19. The method according to claim 18 wherein said step of blanching the edamame product comprises blanching for about ninety seconds.

20. The method according to claim 14, further comprising the step of placing a plurality of the microwaveable bowls in a box, the plurality of the microwaveable bowls having like dimensions and like measurements of the edamame product.
